(19) United States
(12) Patent Application Publication
(10) Pub. No.: US 2011/0130749 A1
(43) Pub. Date: Jun. 2, 2011

(54) METHOD OF ENDOVENOUS LASER TREATMENT OF VARICOSE VEINS

(75) Inventor: Francisco Javier ARCUS VILLACAMPA, Barcelona (ES)

(73) Assignee: INTERMEDIC ARFRAN, S.A., Barcelona (ES)

(21) Appl. No.: 12/626,715

(22) Filed: Nov. 27, 2009

(51) Int. Cl. A61B 18/22

(52) U.S. Cl. 606/15

(57) ABSTRACT

A method of endovenous laser is for treating varicose veins located at a depth below the surface of the skin by the application of laser energy to a selected target region or site.

The method minimizes invasive surgical treatments, causing less perivenous tissue destruction and also less postoperative pain for the patient.

### 1. TECHNICAL FIELD

The present invention relates to methods and procedures for treating varicose veins located at some depth below the surface of the skin by the application of laser energy to a selected target region or site. The finality of the present invention is to minimize the invasive surgical treatments in which a percutaneous or subcutaneous technique in blood vessels is desired. The application of the present invention is basically in the practice of surgery, especially vascular surgery, plastic and cosmetic surgery, as well as dermatology.

### 2. DESCRIPTION OF THE PRIOR ART

The use of laser devices in various types of surgery is well known, and more specifically in vascular disease.

Laser device cause thermal coagulation and/or ablation of tissue by emission of a predetermined level of laser energy for a predetermined time, thus the unwanted tissue can be coagulated to the desired depth if a low energy density is applied, or ablated if a higher level of energy density is applied.

It exists different types of laser techniques especially used in treating larger varicose veins, which are located in the dermal skin layer at depths of up to several millimeters from the skin surface. One of the conventional laser technique is with laser energy delivered from the surface, which requires higher powers that could lead to increased side effects including scarring and skin hyper or hypopigmentation. Other accepted treatments of varicose veins include sclerotherapy, ambulatory phlebectomy, and ligation and stripping of the greater saphenous vein in cases of saphenofemoral junction incompetence.

These traditional surgical therapies are very invasive for the patient, because in some cases general anesthesia is required, and sometimes it can occur possible complications of the surgery, such as bleeding, infection, hypertrophic scars, ankle paresthesia, and a prolonged recovery period.

Recently, more advanced techniques have been developed in order to search for less invasive techniques to treat varicose veins with acceptable short and long term results, which are based on delivering laser energy from below the skin. The most important ones are ultrasound guided sclerotherapy, monopolar electrocautery, a bipolar radio frequency based on energy source delivered by a disposable catheter (VNUS), and lasers techniques. Such techniques have enormous advantages, because they minimize the thermal damage to the skin, and therefore also the possible side effects to the skin.

Conventionally, lasers techniques, which have been used for destruction of blood vessels, have normally operated at wavelengths of slightly below 600 nm. One example is described in U.S. Pat. No. 5,053,033 to Clarke, where laser energy is delivered endoluminally. The subject-matter disclosed in Clarke patent uses laser energy in the range from about 240 to about 280 nm. delivered via an optical fiber or other waveguide incorporated, for example, into a percutaneous catheter. In operation, the ultraviolet laser energy kills smooth muscle cells at an angioplasty site, thereby reducing the risk of restenosis, while minimizing damage to surrounding tissue. However, this technique is used to keep a blood vessel open and, therefore, has little use in the treatment of varicose veins.

In last 5 years, endovenous laser treatment has become a popular minimal-invasive alternative to stripping in the treatment of varicose veins. By catheterizing the varicose vein and introducing a bare fiber into the vein lumen, light energy is delivered intraluminally. This energy is absorbed by blood and water or by the vein wall. The aim of this technique is to obliterate irreversibly the refluxing vein. The typically used diode laser types are the following: 810-940-980 nm diode lasers, and the most commonly used lasers are the 940 and 980 nm lasers, which results are acceptable.

Another disadvantage of the endovenous laser treatment with a diode laser 810 to 980 nm is that the method of treatment is very long, because it involves the step of catheterizing the varicose vein (to facilitate the introduction of the laser energy laser line) before introducing a bare fiber into the vein lumen.

A need, therefore, exists for an improved method for treating varicose veins using a specific energy laser of endovascular laser, in order to make a treatment less damaging (less bleeding and with less edema, erythema and swelling and faster healing than conventional surface laser energy application), more efficient, less painful for the patient, and easier and faster to utilize.

### 2. SUMMARY OF THE INVENTION

The present invention provides a method for treating varicose veins contained at a selected depth and in a selected area of a patient's dermis using a specific laser energy laser of an endovascular laser.

The varicose veins to be destroyed, and in which the laser is positioned, may be specially leg veins, but also telangiectasias, part of a port wine stain, blood vessels at the base of a hair follicle, blood vessels underlying psoriatic plaque, or other subsurface blood vessels, either for health condition or cosmetic reasons.

The method for treating varicose veins comprises the following steps:
Doing an incision or a puncture site.
Introducing from the incision or a puncture site a bare fiber optic line with a rounded edge tip through a vein lumen, advancing through the vein lumen, and positioning the tip of the bare fiber optic line in the selected treatment site in the vein wall.
Once in position, the vein lumen is irradiated with a pulsed mode or in continuous-wave mode laser beam to the selected area, which light has a wavelength of between 1.300 nm and 2.100 nm.
Retracting gradually the bare fiber optic line automatically or manually and operating the laser beam in other selected treatment site in the vein wall, thus (a) when operating with a pulsed light laser beam the doctor retracts the tip of the bare fiber optic line to another treatment site and operates the laser beam and repeats this method various sites along the vessel, and (b) when operating with a continuous-wave laser beam the doctor retracts the tip of the bare fiber optic line and operates the laser beam continuously at the same time that he retracts the bare fiber optic line.
Withdrawn completely the fiber optic line from the vein lumen.
Optionally, after the step of removing completely the fiber optic line, the puncture site and the treated vein can be covered by foam pads, and also additionally a compression bandage can be applied.

The type of laser energy can be selected from either a pulsed or a continuous-wave mode laser, with pulse duration being controlled by gating the laser to provide the pulsed light, or the laser may be a high repetition rate laser with pulse duration again being controlled by gating the laser.

The laser may be operated to deliver a single pulse, or multiple pulses may be provided.

In case of using a pulsed mode laser, each pulse of the pulsed mode energy delivers a pulse duration of between 100 milliseconds and 6 seconds.

In case of using a continuous-wave mode laser, the power and the traction velocity of the laser is programmed to supply energies above the area being treated of between 1 and 300 J/cm (depending on the caliber vein).

In the step of introducing a fiber optic line with a rounded edge tip in a vein lumen, advancing through the vein lumen, and positioning the tip of the fiber optic line in the treatment vein wall to deliver the laser energy, additionally conventional accessories can be used, such as ultrasound guidance or other guiding mechanisms.

The advantageous bare fiber optic line utilized in the present method of treatment comprises: a conventional core, a conventional cladding that recovers the core, and a jacket that recovers the cladding but not right to the tip end, thus the tip end is not covered by the jacket. The tip of the fiber optic line (formed by the core and the cladding only), which is the means for deliver the laser energy into the vessel lumen, is provided with a rounded edge. This rounded edge tip is adapted to minimize the damage of the tip when it is in contact with the wall vein. Such type of damage is especially clear when the vein is large and has twists and turns. Another important feature of this advantageous configuration of the tip is that it permits to eliminate the use of the conventional catheter that is used to positionate the fiber optical line. The diameter of the core of the optical fiber would typically be on the order from 0.2 mm. to 1 mm.

Several experiments have revealed that the preferred range of wavelength of the laser energy is 1450-1550 nm. It has been proved that surprisingly the application of this new wavelength laser (1450-1550 nm. diode laser) generates a high absorption of the energy in water, oxihemoglobin, and in the vessel wall.

Other experiments have conclude that not only direct contact but also light energy absorption by intracellular cytosol of vein wall cells takes part in the mechanism of action of endovenous laser treatment.

Another experiment had compared the energy delivered from the 980 nm diode laser with respect the “high” diode laser (1450-1550 nm.) wavelength laser in the vein wall, and it had concluded that the energy delivered from the 980 nm diode laser is less well absorbed by the vein wall cells and induces more laser energy escapes out of the vein walls, thus increasing the risk of having side effects.

Therefore, on the contrary, using a diode laser of 1450-1550 nm. laser gives less laser energy escapes out of the vein walls, thus reducing the risk of having side effects. Hence, using a diode laser of 1450-1550 nm causes less perivenous tissue destruction and possibly with less postoperative pain.

Three different experiments were carried out to compare a conventional 980 nm diode laser, and the new diode laser 1450-1550 nm. For these specific experiments a diode laser of 1500 nm has been chosen:

### 1. Absorption Test

Two identical sections of saphena vein with the same thickness were positioned on respective supports, and a laser energy source impact perpendicular on the internal wall of the vein, and a laser power meter the number of photons that went through the endothelium.

Results:
Abortion percentage with a 980 nm diode laser: 45%.
Absorption percentage with a 1.500 nm diode laser: 83%.

This test confirm that the 1500 nm beams are better absorbed by saphena sections, and therefore, in order to obtain the same retraction it will be required less energy. On the other hand, due to the less permeability in this range of wavelength, the risk of damaging surrounding tissues will be lower.

### 2. Crepitating Test

When the laser energy hit against the internal surface of saphena vein, it is produced a denaturization of the tissues that it is translated in a quick retraction. This phenomenon becomes evident by a tissue crepitation.

Two different saphena vein samples, and each of these was irradiated with a different wavelength, applying an identical spot and the same application time.

Results:

To obtain the same crepitation (retraction):
Required power with a 980 nm diode laser: 12 W.
Required power with a 1.500 nm diode laser: 1.5 W.

### 3. Irradiated Temperature Test

Two different saphena vein samples, and they were vertically disposed. In the internal wall of the vein, laser energy was applied, whereas the external face temperature was measured by means of an infrared thermometer.

The same energies were applied than the test no. 2.

Results:
Obtained crepitation with 980 nm.: Temperature of the external face: 70° C.
Obtained crepitation with 1.500 nm.: Temperature of the external face: <45° C.

### Conclusions:

The conclusions of these tests are that for a endovenous application, the lengthwave of 1.500 m. permits the same effects than the 980 nm., but with a requirements of power 8 times less. On the other hand, temperature that is achieved immediately after the shot is 35° C. less with the lengthwave of 1.500 m.

There are three preferred embodiments of the tip of the fiber optic line used in the recited method of treatment:

The first embodiment is when the front part of the tip of the fiber optic line, which is formed by the core and the cladding, is completely semispherical.

Another embodiment is when the front part of the tip of the fiber optic line, which is formed by the core and the cladding, is formed by a flat plane in which the whole circumferential perimeter has been made round.

Further another embodiment is when the front part of the tip of the fiber optic line, which is formed by the core and the cladding, is formed by a spherical sector.

And further another embodiment is when the front part of the tip of the fiber optic line, which is formed by the core and the cladding, has a conic configuration tip, and said tip is covered by a glass capsule or sheath. This embodiment has the particularity with respect to the other embodiments that generates a circular irradiation due to the conic configuration of the tip.

Other configurations of the tips of the fiber optic line different from the ones described above could be used with a rounded edge.

### 3. BRIEF DESCRIPTION OF THE DRAWINGS

A preferred embodiment of the method of endovenous laser for treatment of varicose veins and the laser device according to the present invention is described below in detail and by way of a non-limitative example, from which features and advantages thereof will be clearer. The following description is given according to the accompanying drawings, wherein:

FIG. 1 represents a first embodiment of the tip of the bare fiber optic line.

FIG. 2 represents a second embodiment of the tip of the fiber optic line.

FIG. 3 represents a third embodiment of the tip of the bare fiber optic line.

FIG. 4 represents a fourth embodiment of the tip of the bare fiber optic line.

FIG. 5 represents a schematic drawing showing the step of introducing a fiber optic line with a rounded edge tip in a lesser saphenous vein lumen of a leg of a patient.

FIG. 6 represents a schematic drawing showing the step of positioning the tip of the bare fiber optic line with a rounded edge tip through in the selected site in the lesser saphenous vein lumen of a leg of a patient.

### 4. DETAILED DESCRIPTION OF THE PREFERRED EMBODIMENT

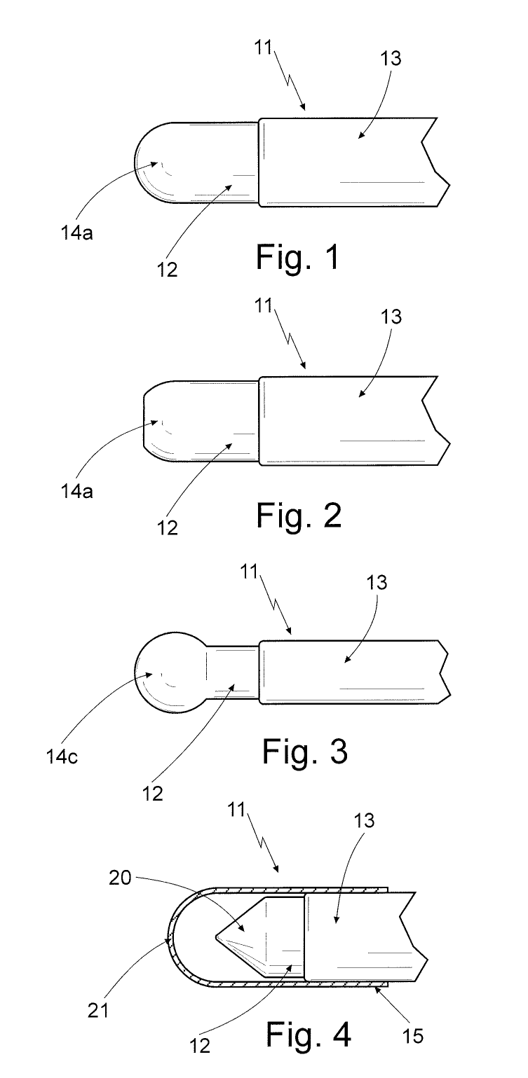

With respect to FIGS. 1-4, there are illustrated different embodiments of the tip of the bare fiber optic line.

All the length of the fiber optic line (10) is formed by a bare fiber optic line which comprises a conventional internal core, a conventional cladding (12) that recovers said internal core, and a external jacket (13) that recovers the cladding (12) but said external jacket (13) finalize slightly before the rounded edge tip of the fiber optic line (11).

The tip of the fiber optic line (11) (formed by the core and the cladding (12) only), which is the means for deliver the laser energy into the vessel lumen (16), is provided with a rounded edge (14a, 14b, 14c), as it is shown in FIGS. 1-3.

In reference to FIG. 4, the core and the cladding (12) have a conic configuration tip (20), and a glass external protector cap (15) that covers the front part of the fiber optic line (11) has a semispherical configuration tip (21).

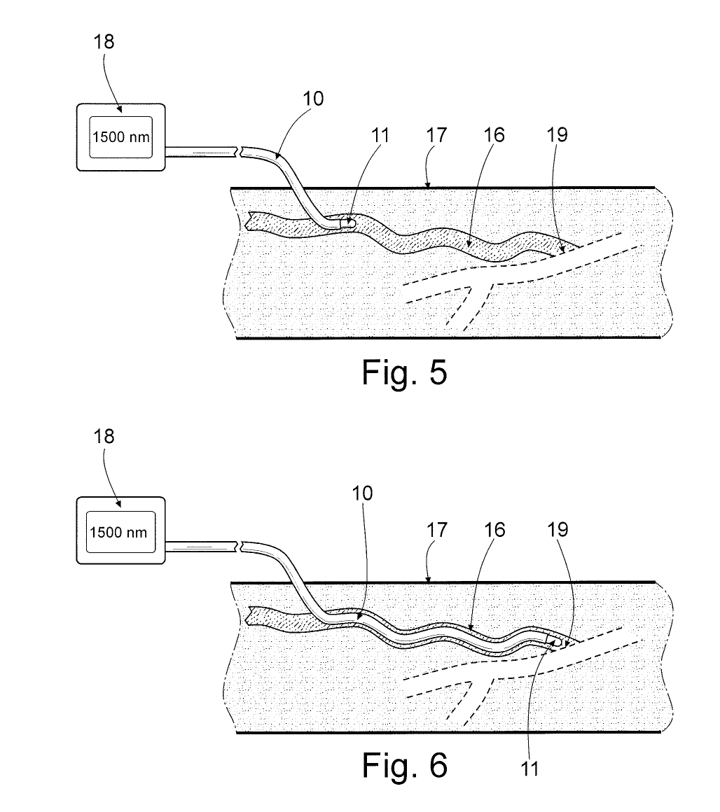

In reference to FIGS. 5 and 6, it is shown two different steps of the claimed method of treatment in a lesser saphenous vein lumen of a leg of a patient.

In FIG. 5 it is shown the introduction of the fiber optic line tip (11) in the lesser saphenous vein lumen (16) of a leg (17) of a patient, through a puncture or an incision site (22). In this case, the puncture or incision site (22) will be located in the maleolar zone.

In FIG. 6 it is represented the positioning of the tip (11) of the bare fiber optic line (10) through the saphenous vein lumen (16) to the selected site in the lesser saphenous vein lumen of a leg of a patient, that in this case is the sapheno-phemoral junction (19).

The guidance of the bare fiber optic line (10) through the saphenous vein lumen (16) is carried out preferably by a first step of eco-marking of the vein path before the treatment; and, after that, when the bare fiber optic line (10) is going through the vein lumen (16) the emission of colored light of the tip (11) that goes through the patient's skin guides optically the doctor. But any catheter is used to guide the bare fiber optic line (10) up to the selected site, because the tip (11) of the bare fiber optic line (10) has an advantageous design. Therefore, the method of treatment is reduced in time and in complicity.

It is clear that other varicose veins could be object of this method of treatment, apart from the described lesser saphenous vein.

In the whole description laser energy has been referred as diode laser energy, but it is easy to understand that other types of laser energy could be used, whenever these changes do not alter of the essence of the invention summarised in the following claims.

1. A method of endovenous laser treatment of varicose veins comprising the following steps:
making an incision or a puncture site;
introducing a bare fiber optic line with a rounded edge tip from the incision or puncture site through a vein lumen, advancing the bare fiber optic line through the vein lumen, and positioning the tip of the bare fiber optic line in a selected treatment site in the vein wall;
once in position, irradiating the vein lumen with a pulsed or continuous-wave mode laser beam, wherein the light of the laser beam has a wavelength between 1.300 nm and 2.100 nm;
retracting gradually the bare fiber optic line and operating the laser beam in another selected treatment site in the vein wall, wherein:
(a) when operating with a pulsed light laser beam, an operator retracts the tip of the bare fiber optic line to another treatment site and operates the laser beam and repeats the method at various sites along the vessel, and
(b) when operating with a continuous-wave laser beam, the operator retracts the tip of the bare fiber optic line and operates the laser beam continuously at the same time that the operator retracts the bare fiber optic line;
completely withdrawing the fiber optic line from the vein lumen.

2. The method of claim 1, wherein the laser beam has a wavelength between 1.450 nm and 1.550 nm.

3. The method of claim 1, wherein in a pulsed mode of the laser energy, each pulse has a pulse duration between 100 milliseconds and 6 seconds.

4. The method of claim 1, wherein in a continuous-wave mode of the laser energy, each pulse delivers a fluence above the area being treated between 1 and 300 J/cm.

5. The method of claim 1, wherein the bare fiber optic line comprises an internal core, a conventional cladding that covers the core, and a jacket that covers the cladding, ending just before the rounded-edged tip.

6. The method of claim 1, wherein a front part of the tip of the fiber optic line formed by the core and the cladding, is semispherical.

7. The method of claim 1, wherein the front part of the tip of the fiber optic line, which is formed by the core and the cladding, is formed by a flat plane in which the whole circumferential perimeter has been made round.

8. The method of claim 1, wherein a front part of the tip of the fiber optic line formed by the core and the cladding, is formed by a spherical sector.

9. The method of claim 1, wherein a front part of the tip of the fiber optic line formed by the core and the cladding, is formed by a conic configuration, and said tip is covered by a glass capsule or sheath.

10. The method of claim 1, wherein after the step of completely withdrawing the fiber optic line, the puncture site and the treated vein are covered by foam pads.

11. The method of claim 10, further comprising applying a compression bandage.
